(19) United States
(12) Patent Application Publication
(10) Pub. No.: US 2008/0237502 A1
(43) Pub. Date: Oct. 2, 2008

(54) System and Method of Identifying Eluant Amounts Supplied to a Radioisotope Generator

(76) Inventor: Frank M. Fago, Mason, OH (US)

Correspondence Address: Mallinckrodt Inc., 675 McDonnell Boulevard, HAZELWOOD, MO, 63042, US

(21) Appl. No.: 11/995,727

(22) PCT Filed: Jul. 26, 2006
(86) PCT No.: PCT/US2006/029055

§ 371 Date: Jan. 15, 2008

Related U.S. Application Data
(60) Provisional application No. 60/702,927, filed on Jul. 27, 2005.

(51) Int. Cl. G21F 5/015; A61N 5/00

(52) U.S. Cl. 250/506.1; 377/21

(57) ABSTRACT

The invention, is directed to a system including a shielded container (16), a radioisotope generator disposed within the shielded container, and an elution supply mechanism. The elution supply mechanism may include an eluant supply container (4) at least partially external to the shielded container (16), a conduit (10) extending between an inlet (20) of the radioisotope generator and an outlet (6, 8) of the eluant supply container, and an eluant visualization portal.

### FIELD OF THE INVENTION

The invention relates generally to the field of nuclear medicine. Specifically, the invention relates to a system and method of identifying an amount or flow of eluant in an elution system configured to enable extraction of a radioactive material from a radioisotope generator for use in the practice of nuclear medicine.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Nuclear medicine is a branch of health science that utilizes radioactive material for diagnostic and therapeutic purposes by injecting a patient with a small dose of the radioactive material, which concentrates in certain organs or biological regions of the patient. Radioactive materials typically used for nuclear medicine include Technetium-99m, Indium-113m, and Strontium-87m among others. Some radioactive materials naturally concentrate toward a particular tissue; for example, iodine concentrates toward the thyroid. However, radioactive materials are often combined with a tagging or organ-seeking agent, which targets the radioactive material for the desired organ or biologic region of the patient. These radioactive materials alone or in combination with a tagging agent are typically defined as radiopharmaceuticals in the field of nuclear medicine. At relatively lower doses of the radiopharmaceutical, a radiation imaging system (e.g., a gamma camera) can provide an image of the organ or biological region that collects the radiopharmaceutical. Irregularities in the image are often indicative of a pathologic condition, such as cancer. Higher doses of the radiopharmaceutical may be used to deliver a therapeutic dose of radiation directly to the pathologic tissue, such as cancer cells.

A variety of elution systems are used to generate radiopharmaceuticals. Unfortunately, radioactive shielding containers of these systems tend to block visualization of the state and progress of the elution process. For example, the amount of available eluant and/or the amount of extracted eluate are generally unknown without opening one or more of the radioactive shielding containers. Rather, the pharmacist typically has to wait an estimated amount of time to ensure the process is complete, which results in wasted time or premature termination of the process. If a specific amount of eluate is desired, then the time estimation may tend to result in too much or too little of the eluate.

### SUMMARY

The present invention, in certain embodiments, is directed to identifying or monitoring a volume, mass, weight, displacement or flow of a supply element (e.g., eluant) and/or an output eluate associated with eluting a radioisotope from a generator product in the field of nuclear medicine. Specifically, in some embodiments, visual access may be provided into an eluant supply container to facilitate performance of elution procedures. For example, a visual portal into an eluant supply container during an elution can provide data for measuring and calculating metrics relating to completion of full or partial elutions and data relating to when a generator is available for milking. Other embodiments may measure an amount or flow of eluant and/or eluate, such that a user can directly view the measurement (e.g., scale or flow meter) or indirectly view the measurement on a remote display screen or computer.

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

In accordance with a first aspect of the present invention, there is provided a system having a shielded container, a radioisotope generator disposed within the shielded container, and an elution supply mechanism. The elution supply mechanism has an eluant supply container at least partially (and in some cases, completely) external to the shielded container, a conduit extending between an inlet of the radioisotope generator and an outlet of the eluant supply container, and an eluant visualization portal.

In accordance with a second aspect of the present invention, there is provided a system that includes a radiation shielded container having a receptacle and a cover disposed over an opening in the receptacle, a radioisotope generator disposed within the receptacle below the cover, and an eluant supply mechanism. The eluant supply mechanism includes an eluant supply container and a conduit coupled with the eluant supply container and the radioisotope generator. The conduit is disposed at least partially within the shielded container, and an eluant measurement device is coupled to the eluant supply mechanism.

A third aspect of the present invention is directed to a method of using a radioisotope elution system. With regard to this third aspect, a radioisotope generator that is disposed inside a radiation shielded container receives an amount of eluant. The amount of eluant received by the radioisotope generator is visually indicated outside the radiation shielded container. In addition, radioactive material is eluted from the radioisotope generator.

In accordance with a fourth aspect of the present invention, there is provided a system including an eluant supply mechanism and a radiation shielded lid having an aperture defined therein. The eluant supply mechanism includes an eluant supply container, a conduit coupled to the eluant supply container and at least partially disposed in the aperture, and an eluant measurement feature.

Various refinements exist of the features noted above in relation to the various aspects of the present invention. Further features may also be incorporated in these various aspects as well. These refinements and additional features may exist individually or in any combination. Again, the brief summary presented above is intended only to familiarize the reader with certain aspects and contexts of the present invention without limitation to the claimed subject matter.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a perspective view of an exemplary embodiment of a generator product including a visually accessible eluant supply bottle, a vented spike, a stop cock, tubing, a shielded lid, a shielded lid plug, and a shielded container;

FIG. 2 is a partial cross-sectional side view of an exemplary embodiment of the generator product, wherein the tubing may pass through an aperture defined along an edge of the lid and into the shielded container;

FIG. 3 is a top view of an exemplary embodiment of a portion of the generator product, wherein the lid may be mounted over an opening in the shielded container;

FIG. 4 is a cross-sectional side view of an exemplary embodiment of the generator product, wherein the tubing may be coupled to the generator via an inlet needle and the lid plug may be replaced by an elution assembly;

FIG. 5 is a partial perspective view of an exemplary embodiment of the generator product, wherein a syringe pump may be incorporated in the place of the eluant supply bottle;

FIG. 6 is a partial perspective view of an exemplary embodiment of the generator product, wherein a drip chamber may be incorporated in the tubing;

FIG. 7 is a partial perspective view of an exemplary embodiment of the generator product that may include the drip chamber, an electronic drop counter, a display, and a computer, wherein the electronic drop counter may be utilized to count the drops passing through the drip chamber;

FIG. 8 is a partial perspective view of an exemplary embodiment of the generator product, wherein the eluant supply may be utilized with a splitter or manifold to supply a plurality of generators, each disposed within a shielded container;

FIG. 9 is a partial perspective view of an exemplary embodiment of the generator product, wherein the eluant supply bottle may be at least partially shielded and may include a visualization window that facilitates viewing and measurement of eluant levels in the bottle, and wherein the drip chamber and drop counter may be disposed within the shielded container; and

FIG. 10 is a partial perspective view of an exemplary embodiment of the generator product, wherein the eluant supply bottle, the drip chamber, and the drop counter may be disposed within the shielded container, and wherein the display may be positioned external to the shielded container along with a portion of a level gauge coupled to the eluant supply bottle.

### DETAILED DESCRIPTION OF SPECIFIC EMBODIMENTS

One or more exemplary embodiments of the present invention are described below. In an effort to provide a concise description of these embodiments, some features of an actual implementation may not be described in the specification. It should be appreciated that in the development of any such actual implementation, as in any engineering or design project, numerous implementation-specific decisions may be made to achieve the developers' specific goals, such as compliance with system-related and business-related constraints, which may vary from one implementation to another. Such a development effort would be a routine undertaking of design, fabrication, and manufacture for those of ordinary skill having the benefit of this disclosure.

The embodiments discussed in detail below relate to a system and method for facilitating efficient extraction of radioactive material (e.g., a radioisotope) from a radioisotope generator during a radioisotope elution process. Indeed, embodiments of the present invention facilitate efficient use of time and resources by providing direct or indirect visual access to an eluant supply and/or an eluate output during a radioisotope elution process. In other words, techniques are disclosed for identifying or tracking a volume, mass, weight, displacement, and/or flow of a supply eluant and/or an output eluate associated with eluting a radioisotope from a radioisotope generator via direct visualization or non-visual measurements that can be visualized remotely. As discussed below, these techniques may include a scale to monitor changes in weight of a supply eluant and/or an output eluate. Additionally or alternatively, these techniques may include a flow meter or displacement gauge, graduated volume marks on the supply and/or output container, and so forth.

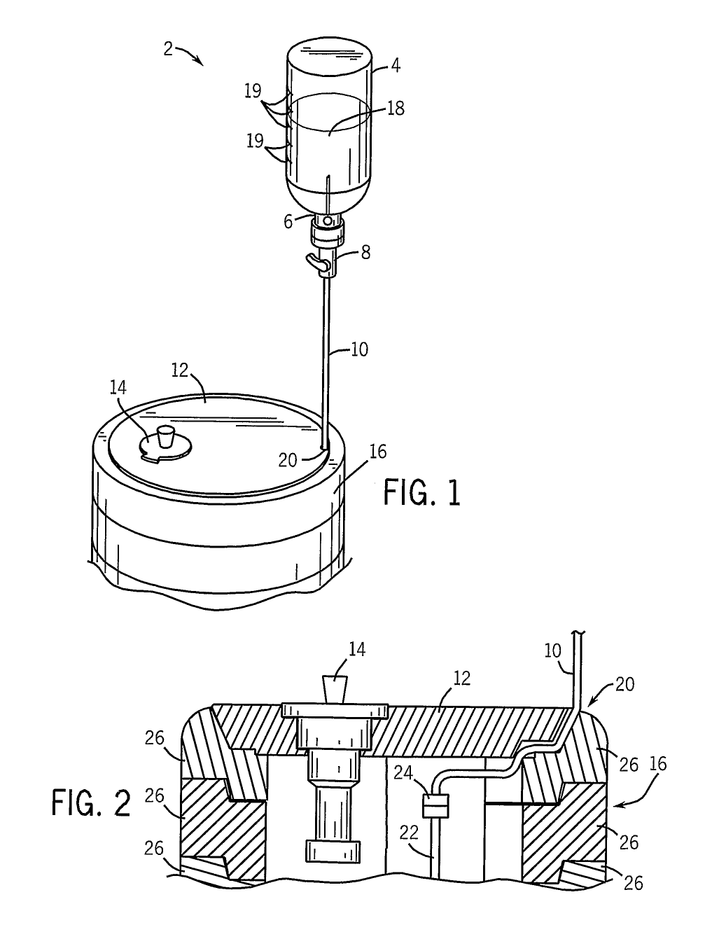

FIG. 1 shows an exemplary embodiment of a generator product 2 that includes a visually accessible eluant supply container (here, a bottle) 4, a vented spike 6, a stop cock 8, tubing 10, a radioactivity shielded lid 12, a radioactivity shielded lid plug 14, and a radioactivity shielded container 16 (e.g., an auxiliary shield). In some embodiments, the lid plug 14 may be replaced by an elution assembly. It should be noted that the term “generator product” herein interchangeably refers to both a radioisotope elution system and/or a radioisotope generator assembly. A radioisotope generator assembly may include a radioisotope generator, a radioactivity shielded container, an eluant supply container, a radioactivity shielded lid, and a lid plug. A radioisotope elution system may include the radioisotope generator assembly, wherein the lid plug is replaced with an elution assembly that includes an eluate output container and an elution shield surrounding the eluate output container.

As illustrated in FIG. 1, the eluant supply container 4 may be entirely or at least partially transparent (or translucent) and external to the shielded container 16, thereby providing a visualization portal into the bottle 4. In some embodiments, the supply bottle 4 may be partially external and/or partially internal to the shielded container 16. The supply bottle 4 can be fully or partially composed of glass, hard plastic, soft plastic, and other appropriate material(s) that allow visual access. As such, a user can visualize eluant 18 disposed within the bottle 4. Because the eluant 18 is visible, a user can observe how much of it has been used during an elution process and/or how much of it remains after an elution process. For example, in the illustrated embodiment, a user can visually monitor the level of eluant in the bottle 4 with respect to index marks 19, which correspond to predefined metrics (e.g., volume). This facilitates determination of when an elution process is complete. Further, if a partial elution (e.g., an elution to partially fill a standard sized eluate output container) is desired, visual access to the eluant supply may facilitate accurate performance of the partial elution. The eluant supply container 4 may be coupled to a generator disposed within the shielded container 16 via the tubing 10. Incidentally, “coupled” or the like herein generally refers to two or more components that are either directly or indirectly connected to one another. In this particular example, the coupling of the eluant supply container 4 and the generator may be characterized as a fluid coupling of those components. Incidentally, “fluidly coupling” or the like refers to a coupling of first and second components so that molecules of a substance(s) (such as a liquid or gas) may be substantially confined within and capable of flowing between the first and second components.

The tubing 10 can be a rigid or flexible conduit (e.g., flexible tubing or a needle) capable of enabling flow of the eluant 18 from the eluant supply container 4 to the generator. In some embodiments, the tubing 10 is transparent and/or translucent, which further facilitates observation of the eluant flow from the eluant supply 18 to the generator. The tubing 10 may be coupled to the eluant supply container 4 in any appropriate manner, such as via a stopcock 8 and a vented spike 6. In the illustrated embodiment, the eluant supply container 4 may be made of a generally rigid material that does not collapse as the eluant 18 is evacuated. Accordingly, the vented spike 6 may allow filtered air to enter into the bottle 4 to reduce the likelihood of a vacuum (e.g., a state of negative pressure) inside the bottle 4 when the eluant 18 flows out. In other embodiments, the eluant supply container 4 may be made of flexible material that collapses as it is evacuated with or without aid by the vented spike 6. The stopcock 8 may enable a user to regulate flow of the eluant 18 from the bottle 4 through the tubing 10 and into the generator. For example, the stopcock 8 may include a valve that opens and closes by means of a tapered plug, enabling a user to control flow of eluant 18 between the bottle 4 and the generator.

The tubing 10 may pass into the shielded container 16 through the lid 12 via an aperture 20 in the lid 12. In some embodiments, the aperture 20 may be formed in a central portion of the lid 12 and may include a nipple or other connection mechanism. However, in the illustrated embodiment, the aperture 20 is disposed along the circumference of the lid 12 such that a gap is formed between the edge of the lid 12 and the shielded container 16. The aperture 20 is illustrated in FIG. 2, which is a partial cross-sectional view of the generator product 2, wherein the tubing 10 passes through the aperture 20 disposed along the edge of the lid 10 and into the shielded container 16. Specifically, FIG. 2 illustrates the tubing 10 passing between the lid 10 and a top section of the shielded container 16 through the aperture 20 and coupling with a generator 22 via a coupling mechanism 24 (e.g., a needle, a nipple, threaded fastener, flange, and/or the like). In some embodiments, the coupling mechanism 24 may include a check valve that reduces the likelihood of backflow of eluant and/or eluate from the generator 22 to the tubing 10 (and possible even the eluant supply container 4). In some embodiments, the tubing 10 may include a check valve disposed therein to reduce the likelihood of backflow from downstream tubing to upstream tubing and/or to the eluant supply container 4. It should be noted that in some embodiments, the tubing 10 may pass through an opening in the side of the shielded container 16. For example, in some embodiments, the tubing 10 may pass through an opening formed between sectional rings 26 that are stacked to form the shielded container 16.

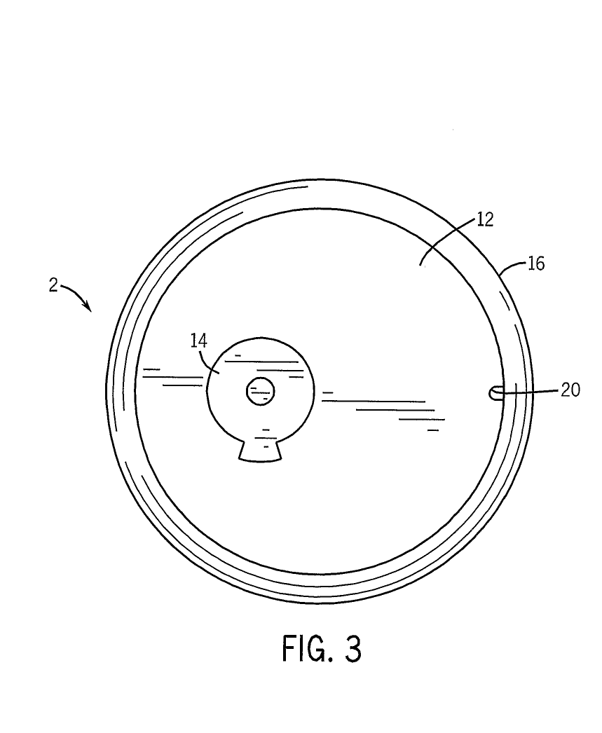

FIG. 3 is a top view of a portion of the generator product 2, wherein the lid 12 is mounted over an opening in the shielded container 16. Specifically, FIG. 3 illustrates the aperture 20 disposed along an edge of the lid 12 and forming a gap between the lid 12 and the shielded container 16. As noted above, in some embodiments, the aperture 20 may be located in a generally central location on the lid 12 or in a side portion of the shielded container 16. In some embodiments, the aperture 20 and the tubing 10 may correspond in size so that the tubing 10 is tightly secured when engaged with the aperture 20. In other embodiments, the aperture 20 may be larger than the tubing 10, allowing maneuverability of the tubing 10 while it is engaged in the aperture 20. In still other embodiments, the tubing 10 includes one or more seals or the like that operate to secure the tubing 10 in the aperture 20 and prevent flow (e.g., air flow) in and out of the shielded container 16 through the aperture 20.

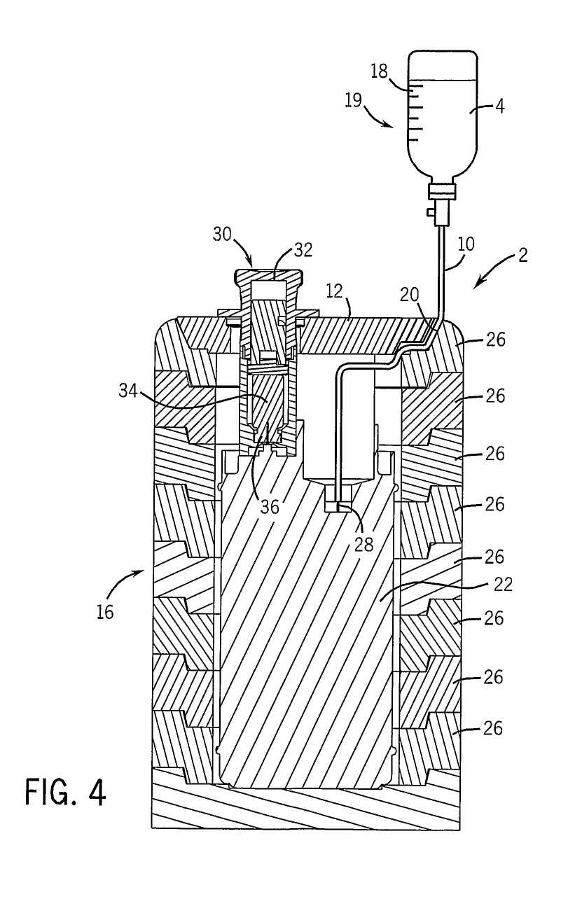

FIG. 4 is a cross-sectional side view of the generator product 2, wherein the tubing 10 is shown coupled to the generator 22 via a hollow inlet needle 28 and the lid plug 14 has been be replaced by an elution assembly 28. The illustrated elution assembly 28 includes an elution shield 32 at least generally disposed about an eluate collection bottle 34. The elution shield 32 is designed to shield users from radioactive elements that are received by elution into the bottle 34. The eluate collection bottle 34 may be coupled to the generator 22 via a hollow outlet needle 36. During a wet elution process (e.g., an elution process wherein the generator generally remains charged), the eluate collection bottle 34 may be coupled to the generator 22 to enable eluate residing in the generator 22 to circulate through the generator 22 and into the evacuated collection bottle 34. The generator 22 is a shielded container that holds a parent radioisotope, such as Molybdenum-99 absorbed to alumina beads or another suitable exchange medium. The daughter radioisotope (e.g., Technetium-99M) is held chemically less tightly than the parent, thereby enabling flowing eluant to flush the desired radioisotope from the radioisotope generator 22 into the collection bottle 34 as eluate.

The eluate collection bottle 34 may have a standard or predefined volume, which may begin in an evacuated condition. A pressure drop into the evacuated eluate collection bottle 34 may facilitate eluate residing in the generator 22 to begin filling the bottle 34. Correspondingly, eluant 18 from the eluant supply container 4 may begin flowing into the generator 22 to replace the eluate passing to the collection bottle 34. Indeed, once the eluate collection bottle 34 is connected to the generator 22, a user can observe that eluant levels in the eluant supply container 4 go down in an amount generally corresponding to the amount of eluate received in the eluate collection bottle 34. For example, a user can observe the volume of eluant 18 leaving the eluant supply container 4 by comparing the eluant level in the supply bottle 4 over time with the index marks 19. This visualization may tend to facilitate determining when the elution process is complete (e.g., the eluate collection bottle 34 is full), and/or may facilitate performance of partial elutions, in which the eluate collection bottle 34 is partially filled with eluate. It should be noted that in some embodiments, the eluate collection bottle 34 may not begin in an evacuated condition. For example, in some embodiments, other system conditions (e.g., generated pressure and/or gravity) may cause flow into the eluate collection bottle 34.

FIG. 5 illustrates an alternative embodiment of the generator product 2, wherein a graduated syringe pump 40 may be incorporated in the place of the eluant supply container 4. The syringe pump 40 is adapted to inject the eluant 18 into the generator 22 via the tubing 10. Because the syringe pump 40 generates pressure, an evacuated eluate collection bottle 34 may or may not be used in this embodiment. For example, a collection bottle 34 with a vent for expelling air may be used to collect the eluate. While the syringe pump 40 may drive the elution, the graduations or volumetric marks 19 may enable a user to measure and/or observe the amount of eluant injected into the generator 22. In other embodiments, other electrical and/or mechanical pumps and measurement systems may be used to supply and measure amounts of eluant supplied to the generator 22. For example, the system may include an electrical/mechanical scale, flow meter, and so forth. Moreover, the measurements may be visualized by a user directly or indirectly via a remote monitoring system, e.g., a computer. It should be noted that FIG. 5 also illustrates that the aperture 20 may be disposed in a generally central portion of the lid 12. Additionally, as shown in FIG. 5, the tubing 10 may be coupled to a nipple 42 that passes through the lid 12 and couples to the generator 22 within the shielded container 16.

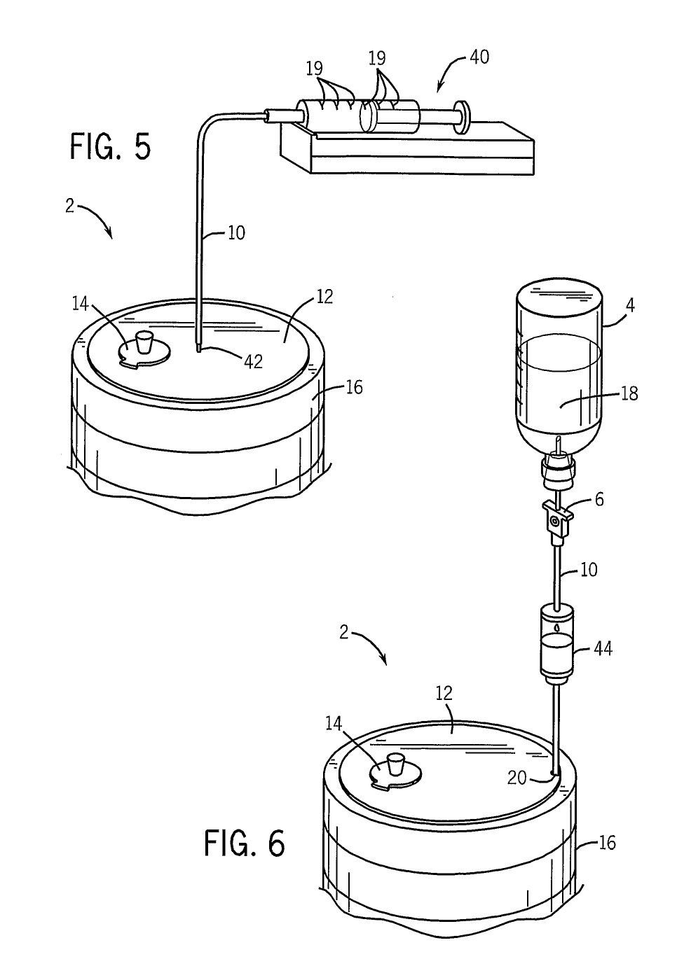

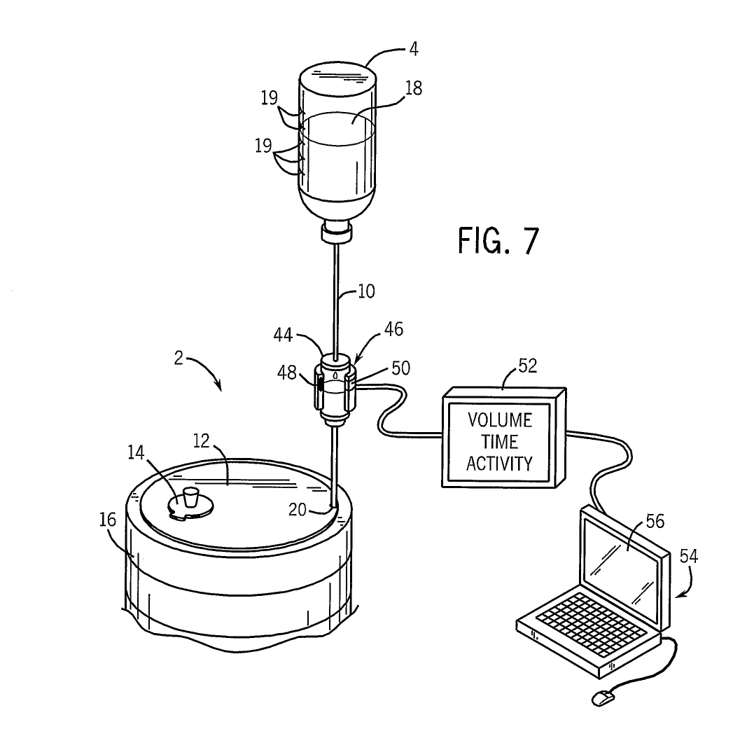

FIG. 6 shows an exemplary embodiment of the generator product 2, wherein a drip chamber 44 is incorporated in the tubing 10 to facilitate tracking or identification of an amount of eluant flowing into the generator 22. The drip chamber 44 may facilitate measurement of the eluant passing between the eluant supply container 4 and the generator 22 in a variety of ways. For example, an observer can manually calculate the amount of transferred eluant by counting the drops that pass through the drip chamber 44. For instance, thirty drops of the eluant may correspond to one milliliter of eluant. As another example, in the embodiment illustrated in FIG. 7, an electronic drop counter 46 may be utilized to count the drops passing through the drip chamber 44 by, for example, detecting motion in the drip chamber 44. In one embodiment, the drop counter 46 may include an infra-red light emitting diode (LED) 48 and a photo detector 50. The LED 48 and photo detector 50 are aligned such that the photo detector 50 receives a light beam from the LED 48. When a drop passes through the drop counter 46, it breaks the light beam and the drop counter 46 outputs and/or stores data corresponding to the break. This facilitates measurement of the number of drops and the provision of metrics relating to the amount of eluant being passed from the eluant supply container 4 through the drip chamber 44 and into the generator 22. Metrics can be calculated from the data retrieved by the drop counter 46 manually, in the drop counter 46 itself, or in other devices capable of receiving data and performing calculations.

As illustrated in FIG. 7, the drop counter 46 may be communicatively coupled to a display 52 for display of metrics relating to the elution process. The drop counter 46 may be coupled to an electronic device and/or computer 54 (e.g., a laptop computer) to store data, facilitate communication with other devices, and/or perform calculations relating to the elution process. It should be noted that in some embodiment, the display 52 may be incorporated into the computer 54. In other words, rather than having a separate display 52, a computer screen 56 of the computer 54 may be utilized for displaying data associated with the elution process. For example, a volume associated with the number of counted drops (e.g., thirty drops corresponds to one milliliter) can be calculated and displayed on the computer screen 56. A time associated with each counted drop can be displayed on the computer screen 56. The volume and/or time associated with each elution process may be tracked and displayed to enable a user (or the computer 54) to estimate when the generator will be ready for another elution process. For example, a value corresponding to an expected radioactivity level of an elution at a certain time can be calculated and displayed on the computer screen 56. By further example, a user (or the computer) can determine an actual radioactivity level of an eluate at a given time. The radioactivity level information can be programmed into the computer 54 if that information is not already in the computer, for example, which can incorporate other data (e.g., time data from the drop counter 46) to determine an expected radioactivity level at a specified future time. In some embodiments, a certain time when an elution should be performed, based on data from the drop counter 46 and/or predefined data (e.g., a calculated expected radioactivity level), can be calculated and displayed on the computer screen 56.

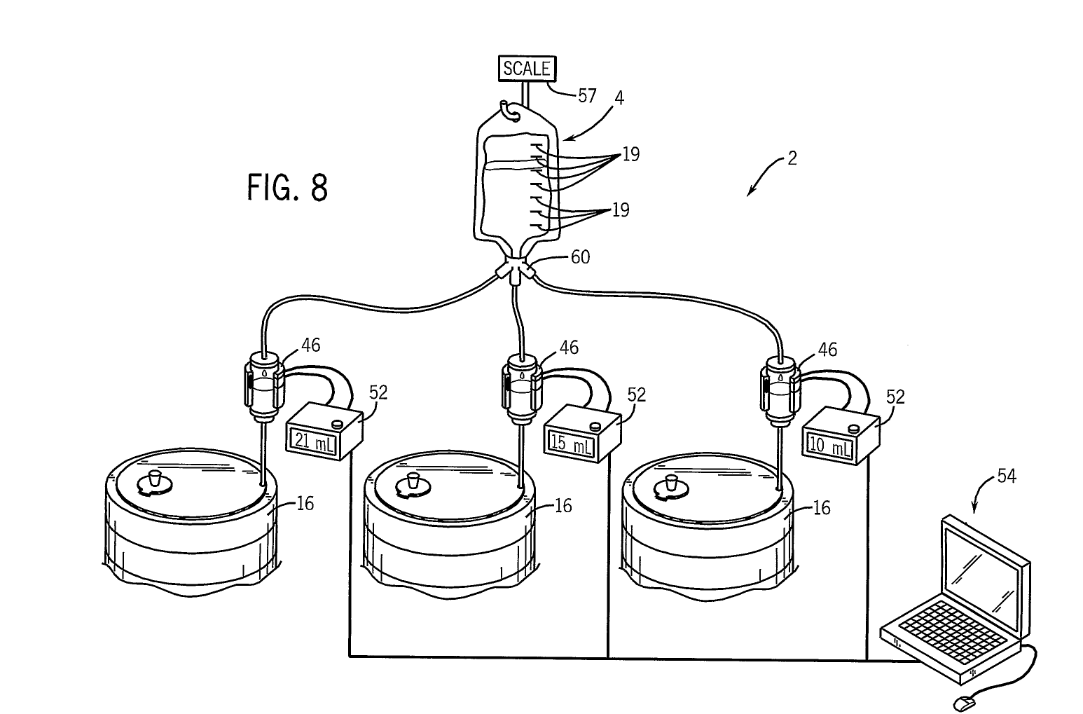

FIG. 8 shows another exemplary embodiment of the generator product 2, wherein the eluant supply container (here, a bag) 4 may be utilized with a manifold or splitter 60 to supply a plurality of generators 22, each disposed within a shielded container 16. As illustrated, this generator product 2 may have a variety of different measurement and visualization features that may complement or supplement one another. The single bulk supply of eluant (e.g., eluant supply container 4) may increase the likelihood that the individual generators 22 have sufficient eluant during individual or simultaneous operation. In addition, the total eluate output from all of the generators may be tracked or visualized by comparing the eluant level inside the bag 4 against the index marks 19.

Still referring to FIG. 8, the computer 54 may be coupled to each of a plurality of drop counters 46 and/or displays 52 that provide data relating to elution processes in each of the generators 22, thus enabling collection and provision of data relating to generator usage individually and/or collectively. For example, based on time stamped usage data and related calculations, the computer 54 may indicate that a particular generator 22 in a set of generators should be milked before the others based on a greater likelihood that it may produce an eluate with an appropriate and/or desired radioactivity level. Further, having a single source of eluant may facilitate rapid replacement of the eluant source (e.g., eluant supply container or bag 4) for multiple generators 22. It should be noted that in the embodiment illustrated in FIG. 8, the eluant supply container or bag 4 may be a transparent or translucent rigid container or a collapsible plastic bag with or without a vent to facilitate flow. Thus, the level of eluant may be directly visualized in the container or bag 4. In some embodiments, the container or bag 4 may be mounted on or hung from a scale 57 to measure weight changes in the container or bag 4 and, thus, track the amount of eluant flowing into the generators. For example, an initial weight of the container or bag 4 may be weighed as a reference, followed by a manual or electronic tracking of reduced weight of the container or bag 4. Alternatively, a separate scale 57 may be attached independently to each of a plurality of eluate supply containers for the generators 22.

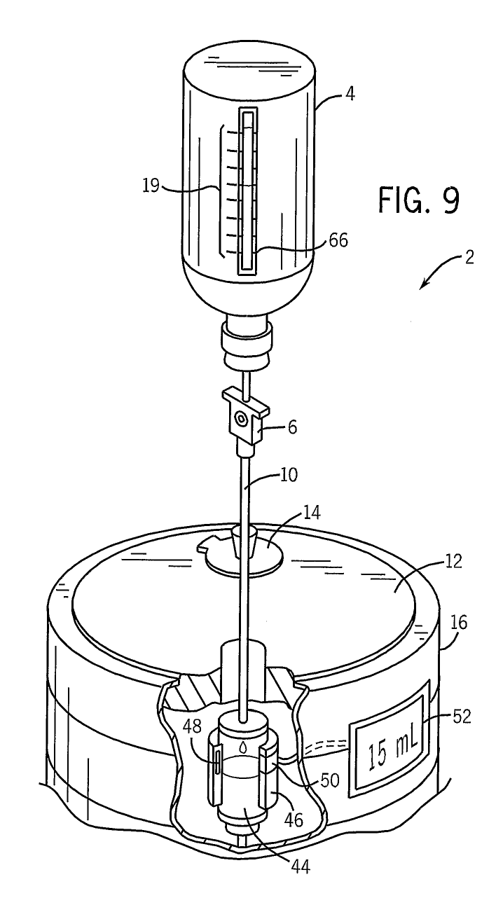

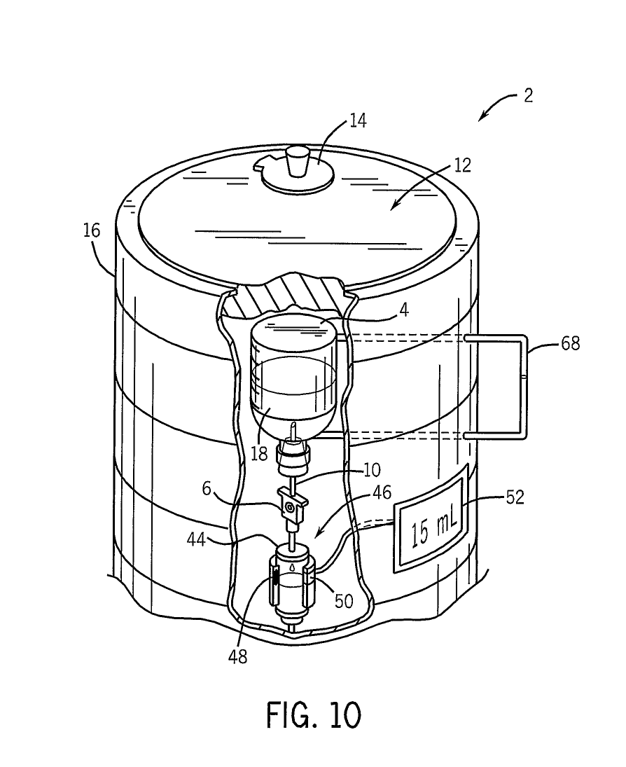

FIG. 9 shows an exemplary embodiment of the generator product 2, wherein the eluant supply container 4 may be at least partially shielded and may include a visualization window 66 that facilitates viewing and measurement of eluant levels in the bottle 4. The window 66 may operate as a visualization portal, which may include index marks 19 that can operate as a measurement feature corresponding to volume or another metric. Further, the illustrated embodiment may include the drip chamber 44 and drop counter 46 disposed within the shielded container 16. Again, the drop counter 46 may be communicatively coupled to the display 52, which may be disposed on the outside of the shielded container 16 to facilitate visual access or identification of the eluant level. Indeed, because the display 52 provides virtual visual access to the eluant supply, the eluant supply container 4 can be disposed within the shielded container, as illustrated by FIG. 10. It should be noted that in FIG. 10 additional access to the eluant level in the eluant supply container 4 may be provided by a level gauge 68 at least partially external to the shielded container. The level gauge 68 can be electronic (e.g., sensor, switches, and electronic display) or manual (e.g., sight glass, circular sight port, or float).

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A radioisotope elution system, comprising:
a radioactivity shielded container;
a radioisotope generator disposed within the radioactivity shielded container; and
an eluant supply mechanism comprising:
an eluant supply container at least partially external to the radioactivity shielded container;
a conduit extending between an inlet of the radioisotope generator and an outlet of the eluant supply container; and
an eluant visualization portal.

2. The system of claim 1, wherein the eluant supply mechanism comprises a drip chamber.

3. The system of claim 2, wherein the eluant visualization portal comprises a transparent or translucent portion of the drip chamber.

4. The system of claim 2, comprising a drop counter coupled to the drip chamber.

5. The system of claim 4, comprising an electronic measurement device communicatively coupled to the drop counter.

6. The system of claim 5, wherein the electronic measurement device comprises a computer.

7. The system of claim 1, wherein the radioactivity shielded container comprises a radioactivity shielded lid having an aperture defined therein, wherein the conduit extends through the aperture in the radioactivity shielded lid.

8. The system of claim 7, wherein the aperture is defined along an edge of the radioactivity shielded lid.

9. The system of claim 1, wherein the radioactivity shielded container comprises a radioactivity shielded lid having a hollow nipple coupled to the conduit.

10. The system of claim 1, wherein the eluant visualization portal comprises a transparent or translucent portion of the eluant supply container having demarcations corresponding to levels of eluant in the eluant supply container.

11. The system of claim 1, wherein the outlet of the eluant supply container comprises a conduit splitter coupled to the conduit and at least one other conduit that leads to a different radioisotope generator.

12. The system of claim 1, wherein the eluant supply mechanism comprises a pump.

13. The system of claim 12, wherein the pump comprises an eluant measurement system.

14. A radioisotope elution system, comprising:
a radiation shielded container comprising a receptacle and a cover disposed over an opening of the receptacle;
a radioisotope generator disposed within the receptacle;
an eluant supply mechanism comprising:
an eluant supply container; and
a conduit coupled with the eluant supply container and the radioisotope generator, the conduit disposed at least partially within the shielded container; and
an eluant measurement device coupled to the eluant supply mechanism.

15. The system of claim 14, wherein the cover has an aperture defined therein through which the conduit extends.

16. The system of claim 14, wherein the conduit comprises a length of flexible tubing.

17. The system of claim 14, wherein the conduit comprises a hollow needle.

18. The system of claim 14, wherein the eluant measurement device comprises an eluant level gauge coupled with the eluant supply container.

19. The system of claim 14, wherein the eluant measurement device comprises a drip chamber.

20. The system of claim 19, wherein the eluant measurement device comprises a drop counter coupled to the drip chamber.

21. The system of claim 14, wherein the eluant measurement device is at least partially disposed inside the radiation shielded container.

22. The system of claim 21, wherein the eluant measurement device comprises a drop counter disposed within the radiation shielded container.

23. The system of claim 14, comprising an electronic display disposed at least partially external to the radiation shielded container and coupled to the eluant measurement device.

24. The system of claim 14, wherein the eluant measurement device comprises a scale.

25. A method of operation for a radioisotope elution system, comprising:
receiving an amount of eluant into a radioisotope generator that is disposed inside a radiation shielded container;
visually indicating an amount of the eluant received by the radioisotope generator, wherein the visually indicating occurs at a location outside the radiation shielded container; and
outputting radioactive eluate from the radioisotope generator.

26. The method of claim 25, comprising calculating a metric based on the amount of eluant received into the radioisotope generator.

27. The method of claim 26, comprising calculating a suggested time for performing a future elution based on the metric.

28. The method of claim 25, comprising creating a time stamp when the amount of eluant is received.

29. The method of claim 25, comprising measuring the amount of eluant received from within the radiation shielded container.

30. The method of claim 29, wherein the measuring comprises counting drops of the eluant.

31. The method of claim 25, wherein the visually indicating comprises electronically displaying a metric of the amount of eluant received.

32. The method of claim 25, wherein the visually indicating comprises providing a visual line of sight to the eluant.

33. The method of claim 25, wherein the measuring comprises weighing the eluant with a scale.

34. A radioisotope elution system, comprising:
a radiation shielded lid having an aperture defined therein; and
an eluant supply mechanism comprising:
an eluant supply container;
a conduit coupled to the eluant supply container and at least partially disposed in the aperture; and
an eluant measurement feature.

35. The system of claim 34, wherein the eluant measurement feature comprises a drip chamber and an electronic drop counter coupled to the drip chamber.

36. The system of claim 34, wherein the eluant measurement feature comprises an eluant visualization portal.

37. The system of claim 34, wherein the eluant measurement feature comprises a scale.

38. The system of claim 34, wherein an output of the eluant measurement device is indicative of the amount of an eluate output from the system.

39. The system of claim 14, wherein the system is configured to determine an output of the radioisotope generator based on an output of the eluant measurement device.
